(19) United States
(12) Patent Application Publication
(10) Pub. No.: US 2026/0090879 A1
(43) Pub. Date: Apr. 2, 2026

(54) HEART VALVE PROSTHESES WITH A DRUG ELUTING MECHANISM

(71) Applicants: NATIONAL UNIVERSITY OF SINGAPORE, Singapore (SG); NATIONAL UNIVERSITY HOSPITAL (SINGAPORE) PTE LTD, Singapore (SG)

(72) Inventor: Theodoros KOFIDIS, Singapore (SG)

(21) Appl. No.: 19/110,415

(22) PCT Filed: Sep. 13, 2023
(86) PCT No.: PCT/SG2023/050621

§ 371 Date: Mar. 10, 2025

(30) Foreign Application Priority Data
Sep. 13, 2022 (SG) 10202250987P

(51) Int. Cl. A61F 2/24; A61K 31/165; A61K 31/337; A61K 31/43; A61K 31/436; A61K 31/545; A61K 31/573; A61K 31/65; A61K 31/675; A61K 31/7048; A61K 31/7056

(52) U.S. Cl. CPC A61F 2/2409; A61F 2/2415; A61F 2/2418; A61F 2/2445; A61K 31/165; A61K 31/337; A61K 31/43; A61K 31/436; A61K 31/545; A61K 31/573; A61K 31/65; A61K 31/675; A61K 31/7048; A61K 31/7056; A61F 2220/0075; A61F 2250/0067

(57) ABSTRACT

A heart valve prosthesis with one or more drug eluting mechanisms embedded in the prosthesis during manufacture.

### BACKGROUND

Prosthetic heart valves have been developed for decades to replace the native valves for propagating blood movement in one direction and prohibiting regurgitation in the other. However, it is widely acknowledged that these prostheses having circular structures primarily consist of biologically inactive, idle components that only provide mechanical function.

The commercially available prosthesis has some disadvantages including structural generation due to calcification, tears, stenosis and pannus formation. Additionally, other concerns relating to the infections and thrombosis may result in severe complications for the host patient. There appears slow progress in the development of the heart valve prosthetic consisting of a drug eluting mechanism.

Therefore, there is a need to provide heart valve prostheses having a drug eluting mechanism that will minimize the risks associated with, inter alia, the calcification, tears, stenosis and pannus formation.

### SUMMARY

There is provided a heart valve prosthesis comprising one or more drug eluting mechanism, wherein said one or more drug eluting mechanism is embedded in the heart valve prosthesis during the manufacture of said heart valve prosthesis.

There is provided a heart valve prosthesis comprising one or more drug eluting mechanism, wherein said one or more drug eluting mechanism is coated onto the heart valve prosthesis during the manufacture of said heart valve prosthesis.

The heart valve prosthesis according to the present disclosure may be selected from the group consisting of a mechanical prosthesis, a biological prosthesis, and a transcatheter prosthesis. The one or more drug eluting mechanism according to some embodiments of the present disclosure may be a slow release, a fast release or a combination of slow and fast release mechanism.

The one or more drug eluting mechanism according to some embodiments of the present disclosure may comprise a drug exhibiting one or more effects selected from the group consisting of antibiotics, anti-calcification, antioxidant, anti-pannus, anti-fibrotic, antiproliferation and combinations thereof. The antiproliferative drug according to some embodiments of the present disclosure may be selected from the group consisting of Sirolimus, Everolimus, Zotarolimus, Biolimus A9, Ridaforolimus, Tacrolimus, Paclitaxel, and Dexamethasone. The antibiotics according to some embodiments of the present disclosure may be selected from the group consisting of Penicillin, Cephalosporin, Tetracyclines, Chloramphenicol, Macrolide antibiotics, Lincomines, Aminoglycoside Antibiotics, Polypeptide Antibiotics, Dichloropyridine, Quinolones, Factor XIa and Plasma Kallikrein (PKa) inhibitors.

For a mechanical prosthesis, the one or more drug eluting mechanism may be coated on strengthening ring or cuff of the mechanical prosthesis or embedded under cuff of the mechanical prosthesis. For a bio-prosthesis, the one or more drug eluting mechanism may be coated on frames or cuff of the bio-prosthesis or embedded under fabric cuff of the bio-prosthesis. For a transcatheter valve prosthesis, the one or more drug eluting mechanism may be coated on frames, cuff or leaflet of the transcatheter valve prosthesis.

In some embodiments, the one or more drug eluting mechanism comprises a drug loaded hydrogel. In some embodiments, the drug loaded hydrogel is formed by (i) injection into a mold of a predesigned shape and (ii) UV crosslinking. According to some embodiments, the predesigned mold is in a shape of an annulus ring, and the annulus ring shaped drug loaded hydrogel is configured to be sewn under a cuff of the mechanical prosthesis.

Optionally, the drug loaded hydrogel comprises drug loaded nanoparticles. Optionally, the drug loaded hydrogel comprises drug loaded nanoparticles. In some embodiments, the drug loaded nanoparticles are Sirolimus-loaded nanoparticles.

Optionally, the drug eluting mechanism comprises nanoparticles, wherein the nanoparticles are liposomes. In some embodiments, the liposomes are composed of Egg Phosphatidylcholine (EggPC), POPC, POPG, DSPC, or any combination thereof.

Optionally, concentration of the drug in the drug eluting mechanism is of a minimum concentration of 80 nM in a total volume of 2 mL of cell culture media.

There is provided a heart repair ring comprises one or more drug eluting mechanism, wherein the one or more drug eluting mechanism is embedded in the heart repair ring during the manufacture of said heart repair ring. The one or more drug eluting mechanism may be embedded in frames or cuff of the repair ring. The one or more drug eluting mechanism may be coated onto the heart repair ring during the manufacture of said heart repair ring. The one or more drug eluting mechanism may be coated onto frames or cuff of the repair ring during the manufacture of said heart repair ring. The heart repair ring may be an annuloplasty ring.

Optionally, the drug eluting mechanism comprises a drug loaded hydrogel. Optionally, the drug loaded hydrogel comprises drug loaded nanoparticles. In some embodiments, the drug loaded nanoparticles are Sirolimus-loaded nanoparticles. Optionally, the drug eluting mechanism comprises nanoparticles, wherein the nanoparticles are liposomes. In some embodiments, the liposomes are composed of Egg Phosphatidylcholine (EggPC), POPC, POPG, DSPC, or any combination thereof.

Optionally, concentration of the drug in the drug eluting mechanism is of a minimum concentration of 80 nM in a total volume of 2 mL of cell culture media.

There is provided a drug eluting mechanism to be incorporated to a heart valve prosthesis for preventing calcification, tears, stenosis, pannus formation or combination thereof.

Advantageously, when a suitable drug eluting mechanism is coated on or embedded in one or more parts of the valve (including annulus wire, frame, stent and cuff), the drug may substantially reduce or inhibit pannus formation thus less structural degradation of the prosthesis. Further, as the drug eluting mechanism may also be coated on the valve annulus, cords and/or leaflets, calcification may be minimized. Less infection may be observed for the prostheses with anti-infection drugs embedded or coated on the surface of the prostheses or part thereof. More advantageously, when the drug is coated on the existing valve implanted in the subject, it may provide a better biocompatibility.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1A is a schematic illustration of a heart valve prosthesis having a drug eluting mechanism in the form of an embedded spring coil, in accordance with embodiments of the present disclosure;

FIG. 1B is a schematic illustration of a spring coil to be embedded in a heart valve prosthesis, in accordance with embodiments of the present disclosure;

FIG. 1C is a schematic illustration of the spring coil as it is embedded within a fabric cuff of the heart valve prosthesis, in accordance with embodiments of the present disclosure;

FIG. 1D is a schematic illustration of a cross section of the spring coil, in accordance with embodiments of the present disclosure;

FIGS. 2A-2B are schematic illustrations of a cross section and a perspective view of a fabric cuff ring, respectively, in accordance with embodiments of the present disclosure;

FIG. 3 is a schematic illustration of a dip-coating method for coating at least a portion of a heart valve prosthesis, in accordance with embodiments of the present disclosure;

FIG. 4A is a schematic illustration of a ring-shaped mold configured for injection of a drug eluting mechanism, in accordance with embodiments of the present disclosure;

FIG. 4B is a schematic illustration of a ring-shaped hydrogel carrying a drug, which was formed by the mold of FIG. 4A, in accordance with embodiments of the present disclosure;

FIGS. 4C-4D are schematic illustrations of the integration of the ring-shaped hydrogel carrying the drug into a fabric cuff of a heart valve prosthesis, and of a complete heart valve prosthesis comprising the ring-shaped hydrogel, respectively in accordance with embodiments of the present disclosure;

FIG. 5 is a schematic illustration of a liposome coated with a positively charged polymer (Poly-L-Arginine), in accordance with embodiments of the present disclosure;

FIG. 6A is a graph illustrating cell growth inhibition of HASMCs (human aortic smooth muscle cells) over 14 days, in accordance with embodiments of the present disclosure; and

FIG. 6B is a graph illustrating cell viability per each of five different Sirolimus concentrations for sirolimus-loaded nanoparticles (SLN) in hydrogel, over 14 days, in accordance with embodiments of the present disclosure.

### DETAILED DESCRIPTION

There is provided a heart valve prosthesis comprising one or more drug eluting mechanism. In some embodiments, the one or more drug eluting mechanism is embedded in the heart valve prosthesis. In some embodiments, the one or more drug eluting mechanism is coated onto a surface in the heart valve prosthesis. In some embodiments, the process of coating or embedding the one or more drug eluting mechanism may occur during the manufacture of said heart valve prosthesis. In some embodiments, the process of coating or embedding the one or more drug eluting mechanism may be undertaken after said heart valve prosthesis is manufactured. It is to be understood that the drug eluting mechanism comprises one or more drugs to be eluted or released to the environment for example a location where the heart valve is implanted.

In some embodiments, the heart valve prostheses include mechanical prostheses, biological prostheses, and transcatheter prostheses. In some embodiments, the prosthesis may include valve repair rings. In some embodiments, the valve repair rings may be mitral valve repair rings. In some embodiments, the heart valve prostheses may be provided with or without stent.

In some embodiments, the biological prostheses include but not limited to porcine and bovine, aortic, mitral, tricuspid, and pulmonary prostheses. In some embodiments, the mitral valve prostheses may be those disclosed in the following patents or publications WO 2017/061956 A1, U.S. Pat. No. 10,709,560 B2, U.S. Pat. No. 11,324,592 B2, WO 2020/214096 A1, US 2020/0237514 A1 and WO 2021/211062 A1.

In some embodiments, the mechanical prosthesis comprises one or more drug eluting mechanism. In some embodiments, the mechanical prosthesis comprises a drug eluting mechanism. In such embodiment, the drug eluting mechanism may be coated on strengthening ring or cuff. In some embodiments, the drug eluting mechanism may be coated on titanium strengthening ring and fabric cuff. In an alternative embodiment, the drug eluting mechanism may be embedded under the cuff to fill the voids under the cuff. In some embodiments, the leaflet of the mechanical prosthesis may be coated by the drug eluting mechanism. In some embodiments, other suitable parts of the mechanical prosthesis may likewise be coated by the drug eluting mechanism.

In some embodiments, the biological prosthesis (or bio-prosthesis) may be porcine and bovine, aortic, mitral, tricuspid, and pulmonary valve prosthesis. In some embodiments, the bio-prosthesis comprises one or more drug eluting mechanism. In some embodiments, the bio-prosthesis comprises a drug eluting mechanism. In some embodiments, the drug eluting mechanism may be coated on triad frames or cuff. In some embodiments, the drug eluting mechanism may be coated on triad frames and cuff. In an alternative embodiment, the drug eluting mechanism may be embedded under fabric cuff to fill the voids under the cuff. In some embodiments, the leaflet of the bio-prosthesis may be coated by the drug eluting mechanism. In some embodiments, other parts of the bio-prosthesis may be similarly coated by the drug eluting mechanism.

In some embodiments, there is provided a mitral valve prosthesis, comprising an asymmetrical, flexible ring, the ring dimensioned to mimic a native mitral annulus of a patient, two leaflets suspended from the flexible ring and configured to coapt with each other, at least two sets of cords, each set of cords attached to a leaflet on a first end and merging into a bundle on a second end, and a drug eluting mechanism coated onto a surface in the flexible ring, leaflets or cords, the flexible ring, leaflets, and cords creating an orifice through which blood flows in one direction. In some embodiments, the drug eluting mechanism is embedded in the flexible ring, leaflets or cords of the mitral valve prosthesis.

In some embodiments, there is provided a mitral valve prosthesis, comprising an asymmetrical, flexible ring, the ring dimensioned to mimic a native mitral annulus of a patient, two leaflets suspended from the flexible ring and configured to coapt with each other, at least two sets of cords, each set of cords attached to a leaflet on a first end and merging into a bundle on a second end, two caps configured to attach to papillary muscles of the patient, each bundle of cords merging into one of the two caps, such that each bundle of cords is connected to the papillary muscles of the patient via one of the two caps, and a drug eluting mechanism coated onto a surface in the flexible ring, leaflets, cords, or caps, the flexible ring, leaflets, cords and caps creating an orifice through which blood flows in one direction, wherein dimensions of the flexible ring, leaflets, cords and caps match imaged dimensions of 3D imaging of a native mitral valve of a heart of the patient and further wherein at least one of said flexible ring, leaflets, cords and caps are fabricated from autologous pericardium of the patient. In some embodiments, the drug eluting mechanism is embedded in the flexible ring, leaflets, cords or caps of the mitral valve prosthesis.

In some embodiments, there is provided a mitral valve prosthesis to be transplanted in a heart, comprises an asymmetrical ring, the asymmetrical ring is dimensioned to mimic a native mitral annulus of a patient, the asymmetrical ring is constructed from a flexible material rolled onto itself towards an outer side of the valve, an anterior flexible leaflet and a posterior flexible leaflet, said anterior leaflet having a convex shape and said anterior and posterior leaflets suspended from the asymmetrical ring and configured to substantially coapt with each other, each of the anterior and posterior leaflets shape is configured to mimic the shape of a native mitral valve, wherein the anterior and posterior leaflets create an orifice through which blood flows in one direction, at least two sets of cords, each set of cords attached to the anterior or posterior leaflet on a first end of the cords and attached at a second end of the cords directly to a cap on a first end of the cap, the cap is configured to be attached onto papillary muscles of the heart on a second end of the cap, and a drug eluting mechanism coated onto a surface in the flexible ring, anterior and posterior leaflets, cords, or caps. In some embodiments, the drug eluting mechanism is embedded in the flexible ring, anterior and posterior leaflets, cords or caps of the mitral valve prosthesis.

In some embodiments, there is provided a mitral valve prosthesis to be transplanted in a heart, comprises an asymmetrical ring dimensioned to mimic a native mitral annulus of a patient the asymmetrical ring is constructed a single piece of flexible material rolled onto itself towards an outer side of the mitral valve, two leaflets made from the single piece of flexible material, at least one of the two leaflets having a convex shape, the two leaflets suspended from the asymmetrical ring, wherein the two leaflets form an orifice through which blood flows in one direction, at least two sets of cords, each set of cords attached to one of the two leaflets on a first end of the cords, and attached into a bundle on a second end of the cords, a cap to be directly connected to the at least two sets of cords on one end of the cap and configured to be sutured onto papillary muscles of the heart on another end of the cap and a drug eluting mechanism coated onto a surface in the flexible ring, leaflets, cords, or caps. In some embodiments, the drug eluting mechanism is embedded in the flexible ring, leaflets, cords, or caps.

In some embodiments, there is provided a mitral valve prosthesis to be transplanted in a heart, comprises an asymmetrical ring, the asymmetrical ring is dimensioned to mimic a native mitral annulus of a patient, the asymmetrical ring comprises least two strands twisted one around the other to construct a coiled coil structure, an anterior flexible leaflet and a posterior flexible leaflet, said anterior leaflet having a convex shape and said anterior and posterior leaflets suspended from the asymmetrical ring and configured to substantially coapt with each other, each of the anterior and posterior leaflets shape is configured to mimic the shape of a native mitral valve, wherein the anterior and posterior leaflets create an orifice through which blood flows in one direction, at least two sets of cords, each set of cords attached to the anterior or posterior leaflet on a first end of the cords and attached at a second end of the cords directly to a cap on a first end of the cap, the cap is configured to be attached onto papillary muscles of the heart on a second end of the cap and a drug eluting mechanism coated onto a surface in the asymmetrical ring, leaflets, cords, or caps. In some embodiments, the drug eluting mechanism is embedded in the asymmetrical ring, leaflets, cords, or caps.

In some embodiments, the transcatheter valve prosthesis comprises one or more drug eluting mechanism. In some embodiments, the transcatheter valve prosthesis comprises a drug eluting mechanism. In some embodiments, drug eluting mechanism may be coated on frames, cuff or leaflet and other components of the valve. In some embodiments, the drug eluting mechanism may be coated on stainless steel frames, fabric cuff or leaflet of the transcatheter valve. In some embodiments, other suitable parts of the transcatheter valve prosthesis may be coated by the drug eluting mechanism.

In some embodiments, there is provided a heart repair ring comprising one or more drug eluting mechanism. In some embodiments, the heart repair ring comprises a drug eluting mechanism. In some embodiments, the drug eluting mechanism may be coated on or embedded in frames or cuff of the repair ring. In some embodiments, the drug eluting mechanism may be coated on or embedded in annuloplasty ring.

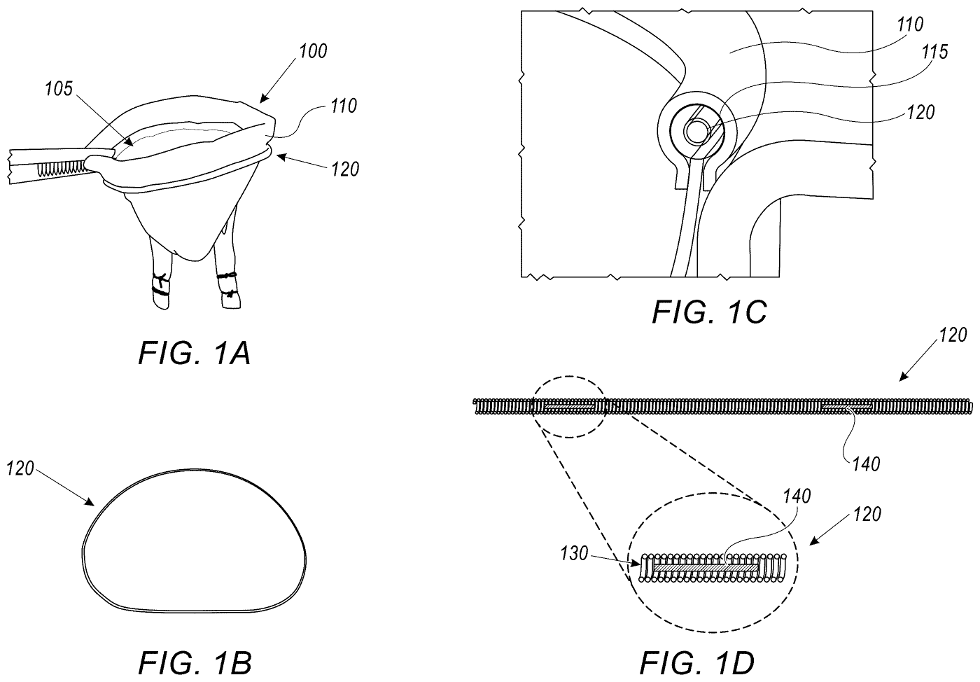

Reference is now made to FIGS. 1A-1D, which are schematic illustrations of a heart valve prosthesis which comprises a flexible annulus wire or spring coil that is configured to be embedded within a fabric cuff of the heart valve prosthesis. As can be seen in FIGS. 1A-1D, heart valve prosthesis 100 may be a mitral prosthesis valve similar to those disclosed in the following patents or publications by the same inventor: WO 2017/061956 A1, U.S. Pat. No. 10,709,560 B2, U.S. Pat. No. 11,324,592 B2, WO 2020/214096 A1, US 2020/0237514 A1 and WO 2021/211062 A1, herein incorporated by reference.

Heart valve prosthesis 100 may comprise a fabric cuff 110, which may be folded and stitched over a spring coil 120 onto annulus 105. Spring coil 120 may provide strength what is the annulus of the heart valve prosthesis 100. In some embodiments, spring coil 120 may be coated by the drug eluting mechanism. In other embodiments, spring coil 120 may comprise a hollow middle 130 which may be configured to carry or be filled with the drug eluting mechanism. Optionally, spring coil 120 may comprise cavities or gaps 140 along spring coil 120, which may be configured to be coated or to carry the drug eluting mechanism. Then, during manufacture of heart valve prosthesis 100, spring coil 120, which may be either coated with the drug eluting mechanism or may carry the drug eluting mechanism along its hollow middle 130, may be positioned along what would become the annulus, once the fabric cuff 110 is folded and stitched over coil spring 120.

In some embodiments, for a heart valve prosthesis 100 having a flexible annulus 105, the flexible annulus wire 120 may be embedded in annulus tunnel 115. In such an embodiment, external fabric cuff 110 may partially or fully cover the annulus 105 and provide space for practitioner to perform suturing in implantation. In some embodiments, the flexible annulus wire 120 may be provided with radiopaque feature. In some embodiments, the flexible annulus wire 120 may be provided in a single, dual or multiple coils. In such embodiment, the drug eluting mechanism may be coated on bovine pericardium or surface of the coil 120. In an alternative embodiment, the drug eluting mechanism may be embedded into cavities 140 in the coil 120. In some embodiments, the drug may be released from surface of bovine pericardium and/or suturing holes due to its low permeability. In some embodiments, the drug may be released in a controlled manner. In some embodiments, the drug eluting mechanism may be coated on leaflet cords that are connected to papillary muscles. In some embodiments, the drug eluting mechanism may be coated on annulus, annulus wire, fabric cuff or other components of the valve including valve cords. For the annulus wire, the drug eluting mechanism may be coated or embedded in hollow annulus wire.

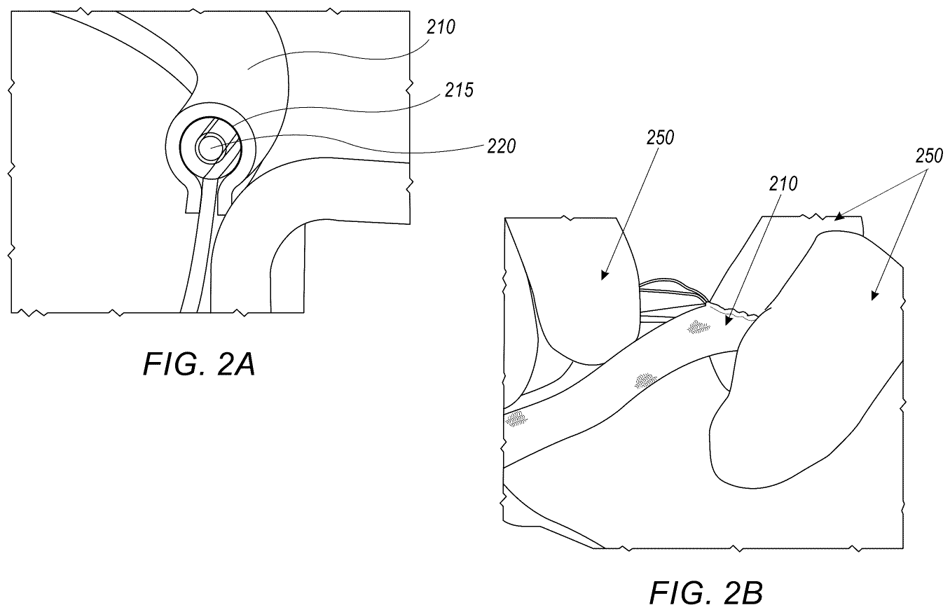

Reference is now made to FIGS. 2A-2B, which are schematic illustrations of a cross section and a perspective view of a fabric cuff ring, respectively, in accordance with embodiments of the present disclosure. According to FIGS. 2A-2B, In some embodiments, for a heart valve prosthesis 200 having a flexible annulus 205, a fabric cuff 210 may partially or fully cover the annulus 205 and provide space for a practitioner to perform suturing in implantation of the heart valve prosthesis 200. In some embodiments, fabric cuff 210 may be coated with the drug eluting mechanism. In such embodiment, the drug eluting mechanism may be coated on bovine pericardium or surface of the fabric cuff 210. In some embodiments, fabric cuff 210 may be folded and sutured such to create an annulus tunnel 215 into which a flexible annulus ring 220 may be inserted by a user, e.g., a physician, illustrated by fingers 250. Flexible annulus ring 220 may be similar to flexible annulus spring coil 120 of FIGS. 1A-1D.

As used herein, the term “drug” refers to an active substance or compound that exhibits at least one of the following effects: antibiotics, anti-calcification, antioxidant, anti-pannus, anti-fibrotic, antiproliferation or combination thereof. Without wishing to be bound by theory, the one or more drugs in the drug eluting mechanism may also include one or more drugs exhibiting other effects than shown above.

In some embodiments, the drug in the drug eluting mechanism embedded or coated may elicit one or more effects selected from the group consisting of antibiotics, anti-calcification, antioxidant, anti-pannus, anti-fibrotic, antiproliferation and combinations thereof. In some embodiments, the drug in the drug eluting mechanism embedded or coated may be statin including but not limited to pitavastatin or pitava.

In some embodiments, the drug is the antiproliferative drug selected from the group consisting of Sirolimus (or Rapamycin), Everolimus, Zotarolimus, Biolimus A9, Ridaforolimus, Tacrolimus, Paclitaxel, and Dexamethasone. Other suitable antiproliferative drug may also be used.

In some embodiments, the drug may be an antibiotic. In some embodiments, the antibiotic may be selected from the group consisting of Penicillin, Cephalosporin, Tetracyclines, Chloramphenicol, Macrolipids, Lincomines, Aminoglycolic Antibiotics, Polypeptide Antibiotics, Dichloropyridine (including 2,4-Dichloropyridine, 3,4-Dichloropyridine, 2,6-Dichloropyridine, and 4,6-Dichloropyridine), Quinolones, Factor XIa and Plasma Kallikrein (PKa) inhibitors.

In some embodiments, the drug eluting mechanism may be provided in the form of various physical phase including but not limited to solid, liquid, semiliquid (a mixture of liquid and solid phases for example slurry) and gel. In some embodiments, one of these vehicles or release mechanisms, whenever applicable may be provided in the rigid or semi-rigid structure.

In some embodiments, the drug release mechanism may be modulated to follow the slow, fast release mechanism or a combination of slow and fast release mechanisms. As will be described below, the release system may be constructed in one or more layers for example outer layer, mid layer, inner layer to allow different release kinetics. In some embodiments, the fast release mechanism may be suitable to address inflammation that typically occurs shortly after the prosthesis is implanted (about one week, two weeks or three weeks after the implantation). On the other hand, the slow release mechanism may be suitable to minimize fibrosis that may occur for example after 7, 8, 9 or 10 years following the implantation. Advantageously, the drug eluting mechanism described herein may also minimize the calcification that may occur within 3-6 months following the implantation. In some embodiments, the calcification may be triggered by pannus. In some embodiments, the calcification may be caused by thrombosis. In some embodiments, the onset of calcification by pannus develops from outer annulus ring inwards.

In some embodiments, when the drug eluting mechanism is coated onto a surface of an element or part of the heart prosthesis, the coating may be provided in a single, double or multiple layer of coating. In the double or multiple layers, different drugs may be provided in the different layers. Any suitable coating techniques may be used for incorporating the drug eluting mechanism to the valve prosthesis including direct coating, coating via crystallization, nano or microporous coating, inorganic porous coating, microporous drug reservoir, nanoparticle coating, drug filling or internal coating and self-assembled monolayers. In some embodiments, the coating layer may comprise core-shell particles, where a first drug is located in the core, partially or fully encapsulated by the shell comprising a second drug, wherein the first drug and second drug are different.

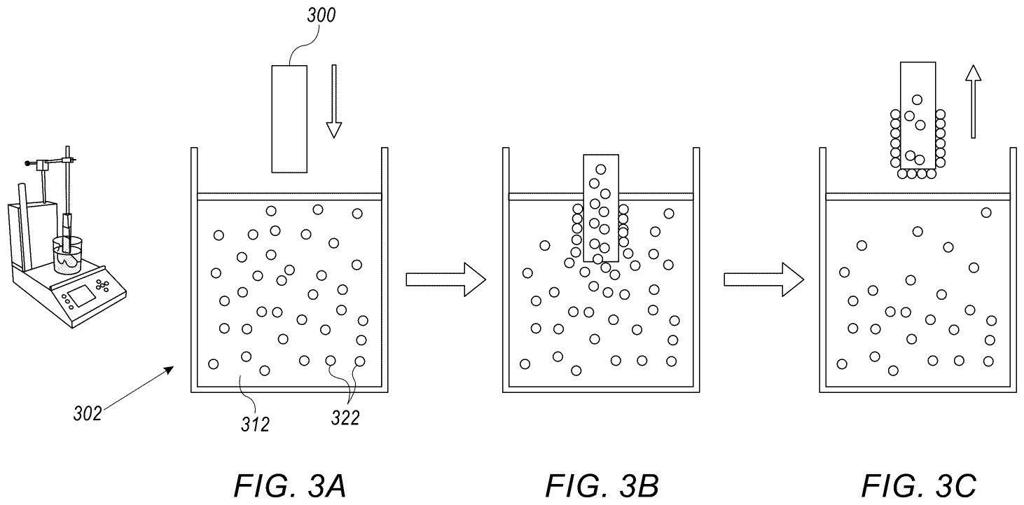

Reference is now made to FIG. 3, which is a schematic illustration of a dip-coating method for coating at least a portion of a heart valve prosthesis, in accordance with embodiments of the present disclosure.

In some embodiments, the drug eluting mechanism may comprise an active substance or a drug that is partially or fully coated or encapsulated. In some embodiments, the coating may be nanocoating (for example nano-spray). The term “nanocoating” used herein refers to a coating process using nanoparticle coating. The nanocoating method may advantageously reduce surface platelet adhesion without detrimental effect on red blood cells. In some embodiments, the nanocoating may comprise polymer. In some embodiments, the polymeric nanocoating may be formed via vapor deposition to advantageously modulate the release kinetics of the drugs thereby improving the therapeutic efficacy of the drug-eluting heart valves. Such modulation may be by adjusting the composition and/or thickness of the nanocoating. In some embodiments, when the drug is one or more antiproliferative drugs, the drug release kinetics may allow evenly distributed release with minimal burst release of the antiproliferative drugs. By incorporating antiproliferative drugs onto the heart valves, this drug eluting device may significantly reduce restenosis in the short term. On the other hand, the long-term therapeutic efficacy of this drug eluting device treatment primarily depends on the control of drug release kinetics. In some embodiments, the active substance or the drug used may suppress the viability and proliferation of human coronary artery smooth muscle cells.

In some embodiments, other suitable coating methods may be adapted to incorporate the drug eluting mechanisms to the prostheses. In some embodiments, the coating may be undertaken via a direct coating method. In such an embodiment, the components or parts of the prostheses for example valve, wire or stent is dipped into a drug solution followed by a solvent evaporation. In an alternative embodiment, the direct coating may be undertaken by dipping components or part of the prosthesis 300, for example, the entire valve or parts of it, e.g., a fabric cuff, an annulus spring coil, a valve leaflet, a wire or a stent into the drug solution 302 using hydrogel precursor matrix 312. In some embodiments, the coating or dip coating may be undertaken using siRNA nanoparticles 322. In some embodiments, the coating comprises coating polymeric materials including monomers of 2-dimethylamino ethyl methacrylic acid (DMAEMA) cross-linked with ethylene glycol diacrylate (EGDA), poly(ethylene) diacrylate (PEGDA) hydrogel, poly(methacrylic acid-co-ethylene glycol diacrylate) or PME, poly(1-dimethylamino acid-co-ethylene glycol diacrylate) or PDE. Other suitable polymeric materials may also be used.

In some embodiments, the coating is not limited to coating parts or components of the prostheses. In some embodiments, the drugs may also be coated onto a surface of internal lumen of the stent or frame or cuff, diffusing through abluminal microholes directly into the vessel wall. In some embodiments, the coating may be undertaken during the manufacture of the prostheses or parts thereof. In such an embodiment, a 3D printing technology may be used. In some embodiments, the 3D printing technology is applied to a stent or frame, for example using graphene-nanoplatelet-doped biodegradable polymer composite. Accordingly, the coating or incorporation of the drug eluting mechanism on the heart prosthesis may be undertaken on the various material used to manufacture the heart valve prosthesis or its components or parts thereof. Non-limiting examples of such material include bovine pericardium, metallic or alloy for example cobalt-chromium (Co—Cr), platinum-iridium (Pt—Ir), platinum-chromium (Pt—Cr), cobalt-nickel (Co—Ni), stainless steel (304 or 316 stainless steel), and fabric made of dacron (polyethylene terephthalate or PET), PolyTetraFluoroEthylene or PTFE. In some embodiments, the fabric may be non-woven fabrics, such as nitinol wire, electrospun materials or elastomeric films/coatings. In some embodiments, the material may be provided as core-shell particle, for example, cobalt-nickel as shell and platinum-iridium as the core. Other suitable core and shell materials may also be used whenever applicable.

In some embodiments, the coating or incorporation of the drug eluting mechanism on the heart prosthesis may be undertaken during the manufacture of the heart prosthesis. In some embodiments, the coating or incorporation of the drug eluting mechanism may be undertaken after the manufacture of the heart prosthesis. Hence, the coating or incorporation method of the drug eluting mechanism may advantageously be applied to commercially available heart valve prostheses.

In some embodiments, the drug eluting mechanism disclosed in the present disclosure, may further comprise a carrier. In some embodiments, the carrier comprises a polymeric material. In some embodiments, said carrier may be a nanocarrier. In some embodiments, the polymeric material may be a crystalline polymer or a mixture of amorphous and crystalline polymer. In some embodiments, the polymeric material may be homopolymer, heteropolymer (including copolymer) or cross-linked polymer. Non-limiting examples of the polymeric materials that may be used include poly(n-butyl methacrylate) or PBMA, BioLinx, poly(lactic-co-glycolic acid) or PLGA, poly(L-lactic acid) or PLLA, Poly(vinylidene fluoride-co-hexafluoropropylene) or PVDF-HF, antisense oligonucleotide, hyaluronic acid, S-Nitroglutathione or GSNO, liposome and graphene. Other suitable carriers not listed above may also be used whenever applicable.

In some embodiments, the drug eluting mechanism embedded or coated may be used to prevent or minimize irregularities (for example inflammation) occurring in a short, medium, or long term after the valve prosthesis is implanted. In some embodiments, the drug in the drug eluting mechanism is selected to prevent or minimize inflammatory that may occur after the valve is implanted (short term, about 1 to 4 weeks). In some embodiments, the drug in the drug eluting mechanism is selected to minimize or prevent fibrosis (long term, about 5 to 10 years). Advantageously, the selected drug may prevent or minimize calcification formed within 3 to 6 months. In some embodiments, the calcification may be caused by pannus. In some embodiments, the calcification is not caused by thrombosis. In some embodiments, the calcification is caused by pannus but not by thrombosis. In some embodiments, the calcification onset develops from outer annulus ring inwards. In some embodiments, when the drug release mechanism comprises Sirolimus, the heart valve prosthesis coated or embedded with sirolimus may be sufficient to provide maximal cellular anti-proliferative effect over one week, two weeks or three weeks.

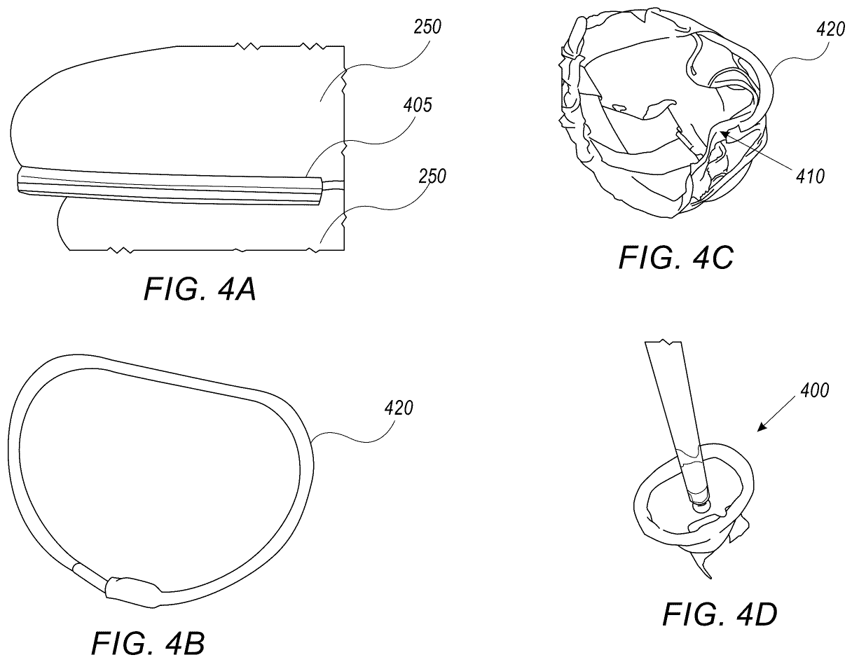

Reference is now made to FIGS. 4A-4D, which are schematic illustrations of manufacturing of a heart valve prosthesis comprising a ring-shaped hydrogel. The ring-shaped hydrogel is configured to include the drug eluting mechanism as well as being embedded within, e.g., inserted into a tunnel created by the folding and suturing of a fabric cuff, as fabric cuff 110 illustrated in FIG. 1A. According to FIG. 4A, a hydrogel loaded with a drug eluting mechanism is injected into mold 405 of a predesigned shape (illustrated as held in between two fingers 250 of a user, e.g., a physician). In some examples, the hydrogel may be loaded with Sirolimus-loaded EggPC Nanoparticles (SLN), though other drug eluting mechanisms may be loaded within the hydrogel. UV is then applied to the injected hydrogel to cause UV crosslinking, and thus cause the hydrogel to harden and be in the form of a gel instead of a liquid. Thus, UV crosslinking is what enables the hydrogel loaded with the required drug, to acquire the shape of the mold 405, e.g., to acquire the form of an annulus wire 420 (FIG. 4B).

The annulus wire shaped hydrogel 420 may then be embedded, e.g., inserted, into a fabric cuff 410 (FIG. 4C). The annulus wire shaped hydrogel 420 may be inserted through a hollow tunnel created by fabric cuff 410, similarly to tunnel 115 in FIG. 1C. Finally, after sewing the fabric cuff 410, a heart valve prosthesis 400 is created, which comprises an eluting drug loaded hydrogel 420.

In some embodiments, incorporating the drug eluting mechanism to the valve prosthesis the drug may be done via nanoparticle coating. In some embodiments, the drug may be Sirolimus, and the nanoparticles may be liposomes composed of Egg Phosphatidylcholine (EggPC).

According to some embodiments, the Sirolimus-loaded EggPC Nanoparticles (SLN) may be synthesized as follows:
i. Desiccate EggPC and Sirolimus for 1 h;
ii. Weigh out 41.7 mg of EggPC and 5 mg of Sirolimus (according to Drug: Lipid mole ratio of 0.1) into separate glass bottle and vial respectively;
iii. Add 1 mL of absolute ethanol to the vial containing Sirolimus and vortex until full dissolved;
iv. Add the solution into the glass bottle containing EggPC and vortex until fully dissolved;
v. Add in excess (minimally 1.2× in excess) of 1.11 mL of PBS in a 3 ml Terumo syringe-aqueous phase;
vi. Add 1 ml of solution containing EggPC, ethanol and sirolimus in a 1 ml Terumo syringe (minimally 1.2× excess of 0.74 ml to account for waste volume)-organic phase;
vii. Nanoparticles are fabricated with the following machine settings:
   a. Syringe: TR 3 ml (dispense 1.11 ml); TR 1 ml (dispense 0.74 ml);
   b. Flow rate ratio: 1.5:1;
   c. Total Volume: 1.85 ml;
   d. Total flow rate: 12.00 ml/min;
   e. Start waste volume: 0.25 mL;
   f. End waste volume: 0.05 mL;
viii. Washing of nanoparticle solution:
   a. Remove the sample falcon tube from the Ignite machine and add 3.7 ml of PBS into the tube (3× dilution-volume of PBS to be added should be 2× the volume of sample collected);
   b. Transfer the solution into an amicon tube;
   c. Place the amicon tube into the centrifuge machine and spin at 3000 ref for 15 mins.
   d. Remove the amicon tube from the centrifuge machine and dispose of the collected filtrate (Note: check that filtrate is clear before disposing-change tube if filtrate is cloudy and include collected filtrate in new tube)
   e. Repeat step c and d until residual solution in amicon tube is less than original sample volume (i.e. 1.5 mL)
   f. Perform another 3× dilution (volume of PBS to be added is 2× the volume of residual solution left in amicon tube) and mix the solution using a pipette
   g. Put the amicon tube back into the centrifuge machine and centrifuge at 3000 ref for 15 mins.
   Repeat step g until residual solution in amicon tube is less than original sample volume (i.e. 1.5 mL).

According to some embodiments, SLN loaded PEGDA hydrogel may be synthesized as follows:
i. Weigh 2.4 mg Irgacure 2959 (12959) and dissolve in 2 mL PBS (1.2% 12959)-vial 1;
ii. Perform serial dilution on nanoparticle solution to obtain final SLN concentration of 80 nM;
ii. Add 275 uL PEGDA 700 MW+225 μL of 1.2% 12959+600 μL of 80 nM SLN into a vial-vial 2;
iv. Pipette 15 μL of solution from vial 2 into a 1 cm silicon mold (from a 8-french catheter tube);
v. Expose loaded silicon mold under UV light (2=365 nm) for 10 mins.

It should be clear that other steps, order of steps, materials, etc. may be implemented instead of the synthesis methods hereinabove.

According to some embodiments, the drug eluting mechanism or drug delivery systems of the present disclosure may be mainly affected, e.g., with respect to controlled drug release rate, by the liposome composition and the molecular weight of the hydrogel.

With respect to liposome composition, experiments have been performed to determine possible optimization. For example, cholesterol of different amounts (e.g., mole ratio) has been added to the EggPC formulation, EggPC has been replaced with 1,2-distearoyl-sn-glycero-3-phosphocholine (DSPC) along with the addition of 50% (mole ratio) Cholesterol, 5% (mole ratio) POPG was added to EggPC/POPC formulation followed by coating of positively charged polymer (Poly-L-Arginine, Chitosan) of a concentration of 8 mg/mL, such that ratio between Drug:POPG:POPC/EggPC is equal to 0.1:0.05:0.95, and finally 5% (mole ratio) DSPG was added to DSPC formulation followed by coating of positively charged polymer (Poly-L-Arginine, Chitosan).

Table 1 summarizes the proposed formulations.

TABLE 1

| | | Mole ratio of Reagents | | |
|---|---|---|---|---|
| No. | Formulation | Sirolimus | Lipid | Cholesterol |
| 1 | EggPC + 15% | 0.1 | 1 | 0.15 |
| | Cholesterol | | | |
| 2 | EggPC + 30% | 0.1 | 1 | 0.3 |
| | Cholesterol | | | |
| 3 | DSPC + 50% | 0.1 | 1 | 0.5 |
| | Cholesterol | | | |
| 4 | EggPC + | 0.1 | 0.95:0.05 | N.A. |
| | 5% POPG + | | (EggPC:POPG) | |
| | PLA coating | PLA coating concentration: 8 mg/mL | | |
| 5 | POPC + | 0.1 | 0.95:0.05 | N.A. |
| | 5% POPG + | | (POPC:POPG) | |
| | PLA coating | PLA coating concentration: 8 mg/mL | | |
| 6 | DSPC + | 0.1 | 0.95:0.05 | N.A. |
| | 5% DSPG + | | (DSPC:DSPG) | |

| | |
|---|---|
| PLA coating | PLA coating concentration: 8 mg/mL |

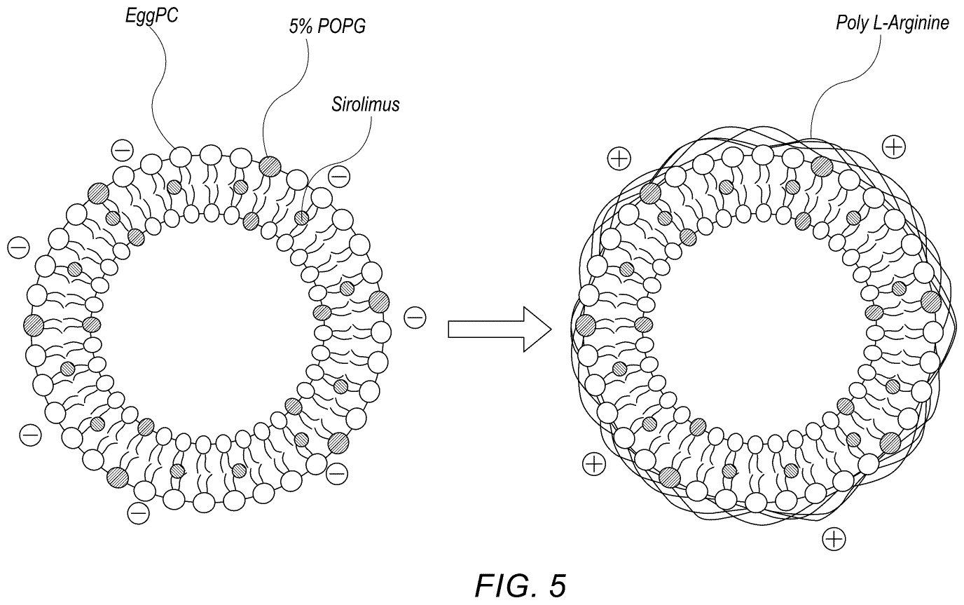

Reference is now made to FIG. 5, which is a schematic illustration of a liposome coated with a positively charged polymer (Poly-L-Arginine), in accordance with embodiments of the present disclosure. As EggPC is an unsaturated lipid, it does not pack efficiently due to the kinks where the carbon double bonds are. The addition of cholesterol allows for a more efficient packing, increases the hydrophobic region for increased amount of encapsulated sirolimus, improves the liposomes' cellular uptake and increases the liposome stability.

Due to the natural origin of EggPC, EggPC degrades relatively fast and is light sensitive. Thus, according to some embodiments, EggPC may be replaced with POPC, which is a synthetic version of EggPC. Another method for improving the stability of the liposome is to coat it with a positively charged polymer (Poly-L-Arginine). Not only does the introduction of a positive charge improve cellular uptake, but the polymer coating also serves as an additional barrier between the encapsulated drug and the external environment, thus helping to slow down liposome degradation to thereby slow drug release.

As illustrated in FIG. 5, some of the EggPC, e.g., 5%, may be replaced with negatively charged POPG. The liposome may carry a drug, e.g., Sirolimus. The Liposome may be coated with a positively charged polymer, e.g., Poly-L-Arginine, to improve stability and control slower drug release, due to slower liposome degradation.

According to some embodiments, EggPC may be replaced with DSPC along with the addition of 50% cholesterol. In comparison to EggPC, DSPC is a saturated lipid with uniform carbon chain lengths of 18 carbons. This creates a larger hydrophobic region for Sirolimus to be encapsulated within. Since DSPC is a saturated lipid without any kinks due to the lack of carbon double bonds, the addition of cholesterol creates spaces and gaps in the hydrophobic bilayer for Sirolimus to be encapsulated within, which is another advantage in addition to the benefit of greater liposome stability.

Encapsulation Efficiency (EE) of the liposome formulations should be significantly higher than 41.5% for formulation with cholesterol or should not be significantly lesser than 41.5% for formulation with Poly-L-Arginine coating.

Table 2 provides microfluidics (ignite machine) fabrication settings for respective formulations.

TABLE 2

| | | Fabrication settings | | |
|---|---|---|---|---|
| | | | Total | |
| | | | Flow | Reagent |
| | | Flow Rate Ratio | Rate? | concentrations/ |
| No. | Formulation | (Aqueous:Organic) | mL/min | mg/mL |
| 1 | EggPC + 15% | 1.5:1 | 12 | Lipid = 41.67 |
| | Cholesterol | | | Drug = 4.95 |
| 2 | EggPC + 30% | 1.5:1 | 12 | Lipid = 41.67 |
| | Cholesterol | | | Drug = 4.95 |
| 3 | DSPC + 50% | 3:1 | 12 | Lipid = 20.84 |
| | Cholesterol | | | Drug = 2.48 |
| 4 | EggPC + | 1.5:1 | 12 | Lipid = 41.67 |
| | 5% POPG | | | Drug = 4.95 |
| | PLA coating | 3:1 | 12 | 8 mg/mL |
| 5 | POPC + | 1.5:1 | 12 | Lipid = 41.67 |
| | 5% POPG | | | Drug = 4.95 |
| | PLA coating | 3:1 | 12 | 8 mg/mL |
| 6 | DSPC + | 3:1 | 12 | Lipid = 20.84 |
| | 5% DSPG | | | Drug = 2.48 |
| | PLA coating | 3:1 | 12 | 8 mg/mL |
| 7 | DSPC + | 3:1 | 12 | Lipid = 20.84 |
| | 5% POPG | | | Drug = 2.48 |
| | PLA coating | 3:1 | 12 | 8 mg/mL |

Table 3 provides the results of fabrication liposomes, that is, which liposomes may be fabricated according to the fabrication requirements in Table 2.

TABLE 3

| Formulations | EE/% | PDI | Size/nm | Charge/mV |
|---|---|---|---|---|
| EggPC + | 41.5 | 0.25 | 77.7 | −0.25 |
| Sirolimus | | | | |
| EggPC + 15% | 53.8 | 0.21 | 99.8 | −1.99 |
| Cholesterol | | | | |
| EggPC + 30% | 55.7 | 0.16 | 91.7 | −1.72 |
| Cholesterol | | | | |
| DSPC + 50% | 54.3 | | 192 | −0.95 |
| Cholesterol | | | | |
| EggPC + 5% | 48.9 | 0.29 | 76.0 | −26.5 |
| POPG + PLA | | | | |
| coating | | | | |
| POPC + 5% | 57.7 | 0.33 | 105.3 | 14.4 |
| POPG + PLA | | | | |
| coating | | | | |

As illustrated in Table 3, a significantly higher encapsulation efficiency (30% increase) was achieved after incorporating 15% of cholesterol, as compared to the proof-of-concept formulation (EggPC+Sirolimus) whilst keeping the particle size (Zeta size) below 100 nm and maintaining the neutral charge. Additionally, the incorporation of POPG (negatively charged lipids) did not significantly affect the Encapsulation Efficiency (EE). With just an addition of 5% POPG, the liposome's charge dropped to −26.5 mV which is desirable as this allows for strong electrostatic forces of attraction with the positively charged polymer coating, e.g., Poly-L-Arginine.

The molecular weight of the hydrogel also have an effect on the drug eluting mechanism per drug release rate, determined e.g., based on swelling ratio.

Table 4 provides different hydrogel compositions per molecular weight (MW), e.g., 700 MW is replaced with 1000 MW, and different blends of molecular weights are provided, of e.g., 700 MW, 1000 MW, 2000 MW and 4000 MW.

The blends of different molecular weights of hydrogel enable to fine tune the mesh size suitable for sustained release of Sirolimus from the liposomes whilst preventing the leakage of the entire liposomes from the gel. The swelling ratio directly correlates to the hydrogel mesh size with PEGDA 3.4 k MW having a mesh size of only 4.51 nm according to literature. When higher molecular weight polymer chains are used for hydrogel synthesis, the distance between the crosslinking points are greater due to the longer linear polymer chains. This translates to a larger mesh size and hence less steric hindrance on only the liposomes housed in the hydrogel. Additionally, there is less inhibition of the drug diffusion pathway out of the hydrogel, thus resulting in faster drug release.

TABLE 4

| Molecular | Total | Standard |
|---|---|---|
| Weight | Avg | Deviation |
| 575 | 1.72 | 0.06 |
| 1000 | 2.72 | 0.09 |
| 4000 | 4.56 | 0.08 |
| 575 (60%)/ | 1.94 | 0 |
| 1000 (40%) | | |
| 1000 (60%)/ | 3.28 | 0.27 |
| 4000 (40%) | | |

Another key selection criteria for the formulations of the drug delivery system is the stability of the nanoparticles as well as extent and duration of anti-proliferation effects on HASMCs. According to some embodiments, the DSPC 50% Cholesterol formulation will provide with a highly stable particle and achieve a highly sufficient sustained anti-proliferation effect. Despite the DSPC 50% Cholesterol formulation not having the highest encapsulation efficiency compared to all the tested formulations, it being a more stable particle allows for slow drug release from within the liposome over a longer period of time, hence resulting in a more sustained anti-proliferation effect. Additionally, by using higher molecular weights PEGDA polymer chains, a hydrogel with larger mesh size is created, to allow for greater amount of drug to elute from the gel during the first few days in order to achieve significant decrease in cell viability within the first 5 days of treatment. Extension of in-vitro experiments to longer than 14 days, e.g., up to 60 days, allows to observe to a fuller extent the difference in the ability of each formulation in achieving different sustained durations of anti-proliferation effect on HASMCs.

For each liposome formulation mentioned above, concentrations starting from 80 nM of sirolimus-loaded liposomes are housed in a 1 cm PEGDA hydrogel of varying molecular weights. Each drug-loaded hydrogel is treated to a population of 30,000 Human Aortic Smooth Muscle cells in a well of a 6-well plate for a duration of up to 60 days. Extent of anti-proliferation capability of each formulation is determined via MTT assay.

Initial proof-of-concept stages have shown that loading of minimally 10 nM of Sirolimus-loaded liposomes into a 1 cm PEGDA (poly(ethylene) diacrylate) hydrogel is able to inhibit the growth of HASMCs (human aortic smooth muscle cells) up to 14 days. The liposomes were composed of Egg Phosphatidylcholine (EggPC). The 700 MW PEGDA was chosen to synthesize the hydrogel, using UV crosslinking method.

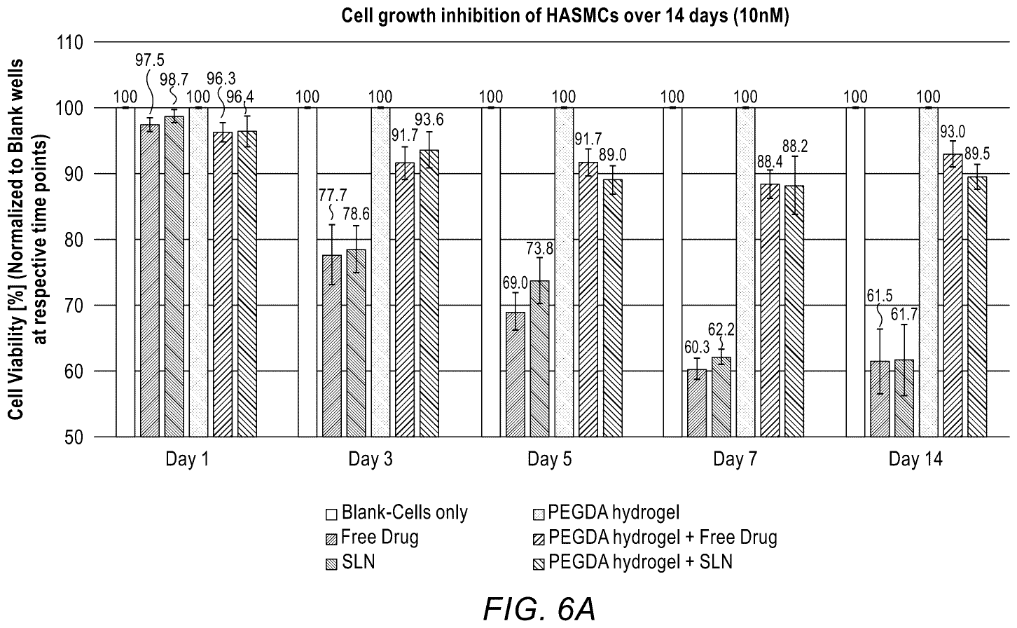

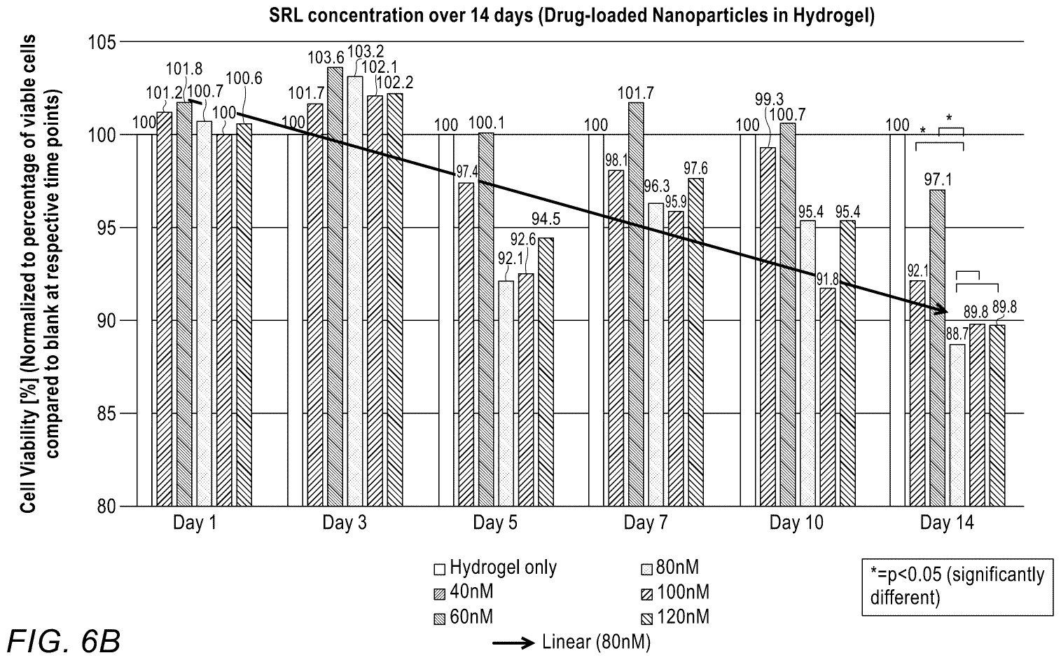

Reference is now made to FIG. 6A, which shows a graph illustrating cell growth inhibition of HASMCs (human aortic smooth muscle cells) over 14 days, in accordance with embodiments of the present disclosure. Cell growth or cell viability was measured per a control that included cells only and no Sirolimus (i.e., Blank). Cell viability is calculated as a percentage of the absorbance of the blank/control wells at a respective time point. The graph in FIG. 6A further illustrates the cell viability of wells with Free Drug (FD), Sirolimus-loaded nanoparticles (SLN), a PEGDA hydrogel with no Sirolimus, a PEGDA hydrogel with FD and a PEGDA hydrogel with Sirolimus-loaded nanoparticles.

The graph in FIG. 6A shows that loading of 10 nM of Sirolimus encapsulated liposomes was able to exhibit anti-proliferation effects on the Human Aortic Smooth Muscle cells up to 14 days, i.e., to inhibit cell growth of the HASMCs to a significant extent for 14 days.

Reference is made to FIG. 6B, which shows a graph illustrating cell viability per each of five different Sirolimus concentrations for Sirolimus-loaded nanoparticles (SLN) in hydrogel, over 14 days, in accordance with embodiments of the present disclosure. According to some embodiments, 3 cm SLN loaded PEGDA hydrogel was treated to population of 30,000 HASMCs in each well of a 6-well cell culture plate. Each hydrogel was directly submerged in cell media with HASMCs attached to the bottom of each well during treatment, for a duration of up to 14 days. At specific timepoints, hydrogels were removed and MTT (3-(4,5-dimethylthiazol-2-yl)-2,5-diphenyltetrazolium bromide) assay was performed to measure cell viability.

As illustrated in the graph in FIG. 6B, Human Aortic Smooth Muscle cells were treated with 5 different concentrations of Sirolimus-loaded nanoparticles (SLN), i.e., 40 nM, 60 nM, 80 nM, 100 nM and 120 nM. It was determined that at day 14, the cell viability in wells treated with 80 nM of Sirolimus is significantly different and lesser (p<0.05) than the cell viability in wells treated with 40 nM and 60 nM of Sirolimus. However, concentrations higher than 80 nM, e.g., 100 nM and 120 nM of Sirolimus showed no significant difference in cell viability after 14 days (p>0.05). Therefore, it may be concluded that 80 nM of Sirolimus (SLN) in a 1 cm/15 μL hydrogel submerged in 2 mL of media, is the minimum drug concentration in a 1 cm PEGDA hydrogel required to achieve maximum anti-proliferation effects for 14 days.

That is, less than 80 nM will not be able to achieve the same maximum extent of anti-proliferation effect on the cells after 14 days. Additionally, for concentrations higher than 80 nM loaded into a 1 cm hydrogel, there is no significant difference in the extent of decrease in cell viability over 14 days. This concentration of Sirolimus is specific for the EggPC liposome formulation loaded into 700 MW PEGDA hydrogel.

According to embodiments of the present disclosure, calculations are based on the total volume of 2 mL of cell culture media in each well. Therefore, 80 nM of Sirolimus in 2 mL of media translates to a concentration of 0.73×10-4 mg/mL.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A heart valve prosthesis comprising one or more drug eluting mechanism, wherein said one or more drug eluting mechanism is embedded in the heart valve prosthesis during the manufacture of said heart valve prosthesis.

2. A heart valve prosthesis comprising one or more drug eluting mechanism, wherein said one or more drug eluting mechanism is coated onto the heart valve prosthesis during the manufacture of said heart valve prosthesis.

3. (canceled)

4. The heart valve prosthesis according to claim 1, wherein said one or more drug eluting mechanism is a slow release, a fast release or a combination of slow and fast release mechanism.

5. The heart valve prosthesis according to claim 1, wherein the one or more drug eluting mechanism comprises a drug exhibiting one or more effects selected from the group consisting of antibiotics, anti-calcification, antioxidant, anti-pannus, anti-fibrotic, antiproliferation and combinations thereof.

6. The heart valve prosthesis according to claim 5, wherein the antiproliferative drug is selected from the group consisting of Sirolimus, Everolimus, Zotarolimus, Biolimus A9, Ridaforolimus, Tacrolimus, Paclitaxel, and Dexamethasone.

7. The heart valve prosthesis according to claim 5, wherein the antibiotics is selected from the group consisting of Penicillin, Cephalosporin, Tetracyclines, Chloramphenicol, Macrolide antibiotics, Lincomines, Aminoglycoside Antibiotics, Polypeptide Antibiotics, Dichloropyridine, Quinolones, Factor XIa and Plasma Kallikrein (PKa) inhibitors.

8. The heart valve prosthesis according to claim 2, wherein the heart valve prosthesis is a mechanical prosthesis, and the one or more drug eluting mechanism is coated on a strengthening ring or cuff of the mechanical prosthesis.

9. The heart valve prosthesis according to claim 1, wherein the heart valve prosthesis is a mechanical prosthesis, and the one or more drug eluting mechanism is embedded under a cuff of the mechanical prosthesis.

10. The heart valve prosthesis according to claim 9, wherein the one or more drug eluting mechanism comprises a drug loaded hydrogel.

11. The heart valve prosthesis according to claim 10, wherein the drug loaded hydrogel is formed by (i) injection into a mold of a predesigned shape and (ii) UV crosslinking.

12. The heart valve prosthesis according to claim 11, wherein the predesigned shape is an annulus ring, and the annulus ring shaped drug loaded hydrogel is configured to be sewn under a cuff of the mechanical prosthesis.

13. The heart valve prosthesis according to claim 10, wherein the drug loaded hydrogel comprises drug loaded nanoparticles.

14. The heart valve prosthesis according to claim 13, wherein the drug loaded nanoparticles are Sirolimus-loaded nanoparticles.

15. The heart valve prosthesis according to any claim 10, wherein the one or more drug eluting mechanism comprises nanoparticles, wherein the nanoparticles are liposomes.

16. The drug eluting mechanism according to claim 15, wherein the liposomes are composed of Egg Phosphatidylcholine (EggPC), POPC, POPG, DSPC, or any combination thereof.

17. The heart valve prosthesis according to claim 9, wherein concentration of the drug in the drug eluting mechanism is of a minimum concentration of 80 nM in a total volume of 2 mL of cell culture media.

18. The heart valve prosthesis according to claim 2, wherein the heart valve prosthesis is a bio-prosthesis, the one or more drug eluting mechanism is coated on frames or cuff of the bio-prosthesis.

19. The heart valve prosthesis according to claim 1, wherein the heart valve prosthesis is a bio-prosthesis, the one or more drug eluting mechanism is embedded under fabric cuff of the bio-prosthesis.

20. The heart valve prosthesis according to claim 2, wherein the heart valve prosthesis is a transcatheter valve prosthesis, the one or more drug eluting mechanism is coated on frames, cuff or leaflet of the transcatheter valve prosthesis.

21. A heart repair ring comprising one or more drug eluting mechanism, wherein said one or more drug eluting mechanism is embedded in the heart repair ring during the manufacture of said heart repair ring.

22-32. (canceled)
